(19) United States
(12) Patent Application Publication
(10) Pub. No.: US 2003/0191347 A1
(43) Pub. Date: Oct. 9, 2003

(54) Venlafaxine base

(76) Inventors: Rolf Keltjens, Nijmegen (NL); Frantisek Picha, Brno (CZ); Juan Cucala Escoi, Barcelona (ES)

Correspondence Address: FLESHNER & KIM, LLP, P.O. BOX 221200, CHANTILLY, VA, 20153, US

(21) Appl. No.: 10/397,381

(22) Filed: Mar. 27, 2003

Related U.S. Application Data
(60) Provisional application No. 60/367,736, filed on Mar. 28, 2002.

(51) Int. Cl. C07C 211/27

(52) U.S. Cl. 564/316

(57) ABSTRACT

Venlafaxine base is obtained in pharmaceutically useful form. Two crystal forms of venlafaxine base are identified and a process of precipitating solid venlafaxine base is provided.

This application claims the benefit of priority under 35 U.S.C. §119 from prior U.S. provisional application 60/367,736, filed Mar. 28, 2002, the entire contents of which are incorporated herein by reference.

### BACKGROUND OF THE INVENTION

The present invention relates to pharmaceutically useful forms of venlafaxine base and methods of making the same.

Venlafaxine is the common name for the compound 1-[2-(dimethylamino)-1-(4-methoxyphenyl) ethyl]cyclohexanol, having the structure shown below.

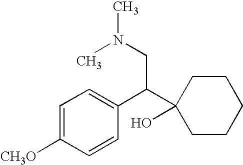

U.S. Pat. No. 4,535,186 describes a class of hydroxycycloalkanephenethyl amines as being useful antidepressants and exemplifies the compound now known as venlafaxine hydrochloride as one of the suitable species. Venlafaxine hydrochloride is approved for sale in various countries including the United States of America. It is available as an immediate release tablet and as an extended release capsule under the brand names EFFEXOR® (Wyeth Ayerst) and EFFEXOR ER® (Wyeth Ayerst), respectively.

Venlafaxine has been the subject of various research endeavors. For example, U.S. Pat. No. 5,043,466 describes a process for making cyclohexanol derivatives in a specified solvent composition. Example 3 of this patent shows the synthesis of venlafaxine as the hydrochloride salt thereof.

U.S. Pat. No. 6,274,171 and related EP 0 797 991A1 disclose encapsulated extended release formulations for venlafaxine hydrochloride. According to these patents, forming an extended release dosage form of venlafaxine hydrochloride was difficult due, in part, to the high water solubility of the hydrochloride salt. The encapsulated dosage form is taught to comprise spheroids of venlafaxine hydrochloride, microcrystalline cellulose, and hydroxypropylmethylcellulose (HPMC).

U.S. Pat. No. 6,197,828 and WO00/32556 discloses the use of individual (+) and (−) enantiomers, respectively, of venlafaxine as well as metabolites thereof. While the commercial venlafaxine hydrochloride is a racemate, these patents teach that various side effects may be reduced by using one isomer substantially without the presence of the other. Resolution of venlafaxine base is achieved by precipitating the di-p-toluoyl-L-tartrate salt or the di-p-toluoyl-D-tartrate salt of venlafaxine followed by neutralization and evaporation of the solvent (ethyl acetate). Similar methods are also described by Yardley et al. in J. Med. Chem. 1990, 33 (10), 2899-2905.

Although venlafaxine hydrochloride provides good pharmaceutical activity, it would be beneficial to find other forms of venlafaxine. In particular, venlafaxine forms that are easier to handle would be advantageous. Venlafaxine hydrochloride is relatively aggressive towards handling equipment and is irritating to the skin, etc., of human personnel that handle the pure active. A venlafaxine form that is less aggressive and less irritating would be desirable. It is further desirable to provide a venlafaxine form that can be easily formulated into various dosage forms including extended release tablets.

While many studies have been reported regarding venlafaxine salts, particularly the HCl salt, and derivatives/metabolites thereof, relatively little is reported regarding venlafaxine base. For example, in U.S. Pat. No. 4,535,186 the free base is made in example 3 but as a mixture of products in the form of an oil. Venlafaxine base is separated via HPLC and the HCl salt precipitated to obtain a solid state form. Similarly, in Example 33, after resolution of the tartrate salt, the free base is obtained by neutralization with NaOH in diethyl ether. The ether is evaporated to leave a solid venlafaxine. The venlafaxine is re-dissolved and precipitated as the HCl salt. U.S. Pat. No. 6,197,828 also evaporates off the solvent (ethyl acetate) to obtain venlafaxine base as a yellow gum that turns into a pale yellow solid (see Example 1). The base is re-dissolved and precipitated as an HCl or tartrate salt. Yardley et al. likewise teach only evaporating off the solvent to obtain a solid form of venlafaxine base. In each of these disclosures, venlafaxine is never precipitated as a free base. Obtaining solid/oily venlafaxine as an evaporation residue generally does not produce a pharmaceutically useful solid form as impurities and solvent are likely to be present. Further, such a method is frequently inconsistent and not easily controllable/reproducible for commercial or industrial scale.

WO 00/76955 discloses enantiomers of O-desmethyl venlafaxine. In making the desmethyl derivatives, example 1 describes the obtaining venlafaxine base as a white solid via evaporation of the CH₂Cl₂ solvent. Example 5 recites precipitating the S(+) enantiomer of venlafaxine base several times from different solvents before conversion to the S(+)-O-desmethyl venlafaxine derivative.

More recently, WO 02/45658 discloses several polymorphs of venlafaxine hydrochloride and their preparation from venlafaxine base. The venlafaxine base is taught to be isolated as a solid via evaporation of the solvent and the resulting residue can be recrystallized from hexane or heptane. FIG. 9 of this disclosure is purported to be a powder x-ray diffraction pattern for crystalline venlafaxine base.

It would be desirable to form racemic venlafaxine base in solid form suitable for pharmaceutical use. It would be further desirable to have a reliable and scalable method for forming such solid racemic venlafaxine base.

### SUMMARY OF THE INVENTION

The present invention is based on the discoveries of how to obtain a pharmaceutically useful, solid form of venlafaxine base and further that venlafaxine base has certain advantageous properties for pharmaceutical formulations and that it can be formed in multiple forms. Accordingly, a first aspect of the invention relates to a crystalline racemic venlafaxine base wherein the venlafaxine is in particle form having an average particle size within the range of 0.5 to 200 microns. Preferably the average particle size is less than 100 microns such as within the range of 10 to 100 microns. The solid particles preferably form a white powder. Typically the pharmaceutically useful form is relatively pure, such as 2.0 wt % or less of impurities, and/or substantially free from solvent, especially ethyl acetate or diethyl ether. Usually the solid is conveniently produced by precipitation of the free base from a solution. In crystalline state, the solid venlafaxine may exist in two forms which are designated herein as Form I and Form II.

An additional aspect of the present invention relates to a crystalline venlafaxine base in particle form having a bulk density of at least 0.4 mg/ml, preferably 0.4-0.8 mg/ml. This particle form preferably is a pure or relatively pure white powder, free from solvents, that is conveniently produced by crystallization. The crystal can be of Form I or Form II or mixtures thereof.

Another aspect of the invention relates to a venlafaxine base in solid form and exhibiting an x-ray diffraction pattern that includes peaks at angles of 2θ of 12.8, 13.3, 18.9, and 20.0 degrees +/−0.2, said peaks having an intensity of at least 10% of the maximum intensity. Preferably this solid venlafaxine is pure or substantially pure venlafaxine base Form II, as is hereinafter described. Related to this aspect of the invention is a venlafaxine solid, comprising a mixture of crystalline venlafaxine base of Forms I and II.

A further aspect of the present invention relates to the discovery of how to precipitate venlafaxine base. In particular the present invention includes a process for making solid racemic venlafaxine base, which comprises precipitating racemic venlafaxine base from a solution of venlafaxine to form crystalline racemic venlafaxine base; wherein the precipitation is carried out in accordance with at least one of the following conditions:

(i) the solution contains a combination of solvents comprising at least one organic polar solvent and at least one contrasolvent selected from water, an aliphatic hydrocarbon and an alicyclic hydrocarbon; or

(ii) a seeding crystal of venlafaxine base is present. The method can be carried out under one or both of conditions (i) and (ii). Preferably the method produces precipitated venlafaxine base having an average particle size within the range of 0.5 to 200 microns and at least 50% of the particles are within the range of +/−25 microns from the average particle size.

An additional aspect of the invention relates to a process for making crystalline venlafaxine base of bulk density of at least 0.4 mg/ml, which comprises crystallizing venlafaxine base from a solvent while cooling, wherein the cooling rate is not greater than 20° C./hour, preferably about 10° C./hour.

### BRIEF DESCRIPTION OF THE DRAWINGS

FIG. 1 shows the powder X-ray diffraction pattern for venlafaxine Form I.

FIG. 2 shows the powder X-ray diffraction pattern for venlafaxine Form II.

### DETAILED DESCRIPTION OF THE INVENTION

The present invention is based on the surprising discovery that a solid, pharmaceutically useful form of venlafaxine base can be formed. In the above-described prior literature, a solid venlafaxine base was normally formed by evaporating off the solvent to leave a solid residue. The solids produced in this way were generally not in a pharmaceutically useful form as they contained the residue of solvent and impurities as indicated by the yellow color and/or color changes associated therewith. Instead of precipitating a racemic free base directly, the above-described patents usually teach converting the free base to a salt, such as the hydrochloride or tartrate salt, in order to accommodate/facilitate precipitation. The present invention provides a method for precipitating venlafaxine base directly. Via precipitation, alone or with other processing techniques known in the art, a solid pharmaceutically useful venlafaxine base can be obtained.

Furthermore, the invention is based on the subsequent discovery that venlafaxine base exhibits low water solubility and slow dissolution in water, which combined with its high loading capabilities, makes it an advantageous active for formulating into pharmaceutical dosage forms, especially extended release dosage forms. In addition, venlafaxine base is less aggressive, less irritating, and easier to handle than venlafaxine hydrochloride. Accordingly, venlafaxine base is easier to formulate into a variety of dosage forms, especially extended release dosage forms, than venlafaxine hydrochloride.

“Venlafaxine base” as used herein means the compound 1-[2-(dimethylamino)-1-(4-methoxyphenyl) ethyl]cyclohexanol and includes the racemate or mixture of enantiomers of venlafaxine as well as the pure or substantially pure (+) or (−) enantiomer of venlafaxine (hereinafter sometimes referred to as (+)-venlafaxine and (−)-venlafaxine).

A “pharmaceutically useful form” as used herein means that the venlafaxine solid is of such a grade as to be suitable for use in a pharmaceutical formulating method and/or for inclusion into a pharmaceutical composition; i.e. ready for such pharmaceutical manufacturing steps as blending, mixing, or milling, etc. The form needs to be susceptible of reliable and reproducible manufacture. As a pharmaceutical active ingredient, the solid venlafaxine base should be relatively pure, typically 5.0 wt % or less, preferably 2.0 wt % or less, more preferably 1.0 wt % or less, still more preferably 0.5 wt % or less of impurities. The solid should be substantially free of solvent, especially ethyl acetate or diethyl ether. Typically the amount of solvent is 3 wt % or less, preferably 1 wt % or less, more preferably 0.5 wt % or less.

The solid form is preferably a white powder. The lack of color in comparison to the prior art evaporate residues indicates that coloring impurities and/or solvents are absent or sufficiently minimized to afford a white color. Preferably the white color meets or exceeds the B9 standard as determined by Pharmacopieal Europe test 2.2.2 (i.e., 0.1% solution in 1% aqueous HCl test).

The solid form is preferably in the form of particles and in particular crystals. The particles are preferably “filtratable,” meaning that the particles can be separated from a solvent by use of a filter. Generally, the particles must be at least 0.5 microns on average to be filtratable. The particle population typically has an average particle size within the range of 0.5 to 200 microns, more typically 10 to 100 microns. Such population is useful and advantageous e.g. for wet granulation or for hot melt granulation. In some embodiments, such as for use in a wet granulation processes, the average particle size is preferably within the range of 1 to 50 microns, more typically 5 to 50 microns, and preferably 10 to 50 microns. In other embodiments, such as for use in direct compression tabletting processes, the average particle size is within the range of 50 to 150 microns, more typically 50 to 100 microns. A second preferred particle size population is from 200 to 500 microns, more preferably 250 to 500 microns. Such population is useful for direct compression into tablets containing high relative amount of venlafaxine.

Crystalline venlafaxine base in particle form preferably has a bulk density of at least 0.4 mg/ml, preferably 0.4-0.8 mg/ml. A population of particles having this bulk density are convenient to handle, having generally good flow characteristics and can be advantageous in formulating into pharmaceutical compositions, especially tablets and more especially tablets made by direct compression. A bulk density of at least 0.4 mg/ml generally, though not always, means that the average particle size is fairly large, typically up to 500 microns. The population preferably is comprised of particles having the above-described average particle size, especially average particle sizes within the range of 250 to 500 microns. However, such an average particle size is not required to meet the recited bulk density range as the bulk density also depends in part upon the shape of the particles. Thus, regardless of the particle size, a bulk density of at least 0.4 mg/ml is a preferred embodiment of venlafaxine base.

Conveniently the solid venlafaxine base of the present invention is a precipitate, especially a filtratable precipitate. By precipitating venlafaxine base from a venlafaxine solution, a solid form is obtained that is relatively pure and, after isolation from the liquid, typically of white color. Such a solid is preferably of such quality as to be a pharmaceutically useful form. The precipitation according to the present invention is preferably “induced” in that a contrasolvent or seeding crystal, or both, is present/added to cause precipitation instead of merely decreasing temperature or solvent volume, albeit such steps may and usually are additionally carried out as well. Induced precipitation is preferred as it generally provides for better yields and/or more economical or practical conditions than simply lowering the temperature, although a technique can result in precipitation or crystallization. As used herein, the term “venlafaxine solution” means any solution that contains a venlafaxine moiety or ion thereof and specially includes venlafaxine salt solutions as well as crude venlafaxine base solutions including the reaction medium obtained by synthesizing venlafaxine. The venlafaxine solution contains a venlafaxine solvent sufficient to dissolve the venlafaxine. Generally organic polar solvents are suitable to dissolve venlafaxine. Specific examples include esters such as ethyl acetate, ethers such as diethyl ether, ketones such as acetone, and lower alcohols such as methanol, ethanol, and 1- or 2-propanol. Contrasolvents are liquids in which venlafaxine base is less soluble than the solvent. Examples include water and aliphatic or alicyclic hydrocarbon solvents such as hexane, heptane, petroleum ether, and cyclohexane. The following combinations are preferred, ethyl acetate solvent and n-heptane contrasolvent; ethanol solvent and n-heptane contrasolvent; and ethanol as solvent and water as a contrasolvent. In the combinations comprising water, it is also preferred that a venlafaxine base seeding crystal is added.

The venlafaxine base is precipitated from the venlafaxine solution to form a suspension, distribution, and/or slurry of solid venlafaxine base in the remaining liquid. Preferably the solid venlafaxine is homogeneous in that the solid particles are of relatively uniform size, i.e. at least 50%, more preferably at least 60%, of the particles are within the range of +/−25 microns from the average particle size. The solid particles, normally crystals, are preferably separated from the solution by filtration. The particles can be washed and/or dried. If desired, the particles can be re-precipitated by dissolving them in a venlafaxine solvent to form a solution and carrying out induced precipitation again.

The venlafaxine solution can be formed by a variety of ways and is not particularly limited in this respect. One route to obtain a venlafaxine solution involves forming a solution of a venlafaxine salt such as venlafaxine hydrochloride or venlafaxine besylate. The salt is usually in solid state form and dissolved in a solvent to form a solution, although such is not required.

The venlafaxine salt solution is typically neutralized by adding an organic or inorganic base, typically NaOH, to form a venlafaxine base solution before inducing precipitation. Suitable bases useful in the neutralization process are alkali metal or alkaline earth metal or ammonia cations, such as those in sodium, potassium or calcium hydroxide, sodium or potassium carbonate, or ammonium hydroxide. Further suitable bases include an organic amine, such as methylamine, triethylamine or pyridine. The mutual ratio of the substrates is preferably approximately stoichiometric (equimolar), however slight molar excess of either component, particularly the base, is not excluded. The solvent system is preferably monophasic, i.e. it comprises a single solvent or a mixture of mutually miscible solvents. Advantageously, the solvent system is so selected that the starting venlafaxine salt and the neutralizing base are soluble in the solvent system, at least at an elevated temperature, but this is not required. Suitable solvent system comprises water and mixtures of water with water-miscible organic solvents such as lower aliphatic alcohol (methanol, ethanol), ketone (acetone, methyl isobutylketone) or cyclic ether (dioxan, tetrahydrofuran).

In an advantageous mode, venlafaxine salt is dissolved or suspended in one part of the solvent system and a solution or suspension of the base in another part of the solvent system is added thereto portionwise until the reaction is completed. The composition of both parts of the solvent system may be identical or different. Completion of the neutralization reaction may be monitored, e.g. by measuring pH, the optimum value being of about 6 to about 9, more preferably 8-9. The temperature of contacting may be ambient, but the reaction mixture may be also heated, optionally up to the reflux, and then cooled after the reaction is completed. The contrasolvent may be a part of the solvent system or may be added after the neutralization reaction is completed. If seeding crystals are used, they are normally added after completion of neutralization.

Alternatively, the neutralization process can use a solvent system that is biphasic. The neutralization reaction proceeds in a first, essentially aqueous phase and the product of the reaction is extracted into the second phase, immiscible with the first, while the rests of the reagents and the salt co-product remains in the first phase. After separation of the phases, the venlafaxine base is precipitated from the solution in the second phase as described above.

Another way to form a venlafaxine solution is from a crude venlafaxine base. The crude venlafaxine solution can be made by dissolving in a venlafaxine solvent a previously formed solid venlafaxine base, especially non-precipitated solid venlafaxine formed by evaporation of the solvent. Alternatively, the crude venlafaxine solution can be the extracted venlafaxine product from a reaction medium. Further, the crude venlafaxine solution can be the entire reaction medium resulting from the synthesis of venlafaxine.

A conventional prior art processes for making venlafaxine comprise, in its last step, a conversion of a suitable precursor of a basic structure (2) into venlafaxine.

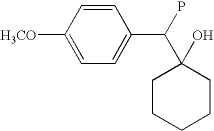

The precursor (2) is a compound having a group P that is convertible into dimethylaminomethyl group of venlafaxine. An example of such a group P is an aminomethyl-group and the conversion comprises N-methylation the corresponding aminomethyl-precursor by aqueous formaldehyde and formic acid according to a modified Eschweiler-Clarke reaction (see the above cited Yardley et al. J. Med. Chem., Method A). Another example of P is dimethylaminocarbonyl group that is convertible to the desired dimethylaminomethyl by reduction in a tetrahydrofuran solution with electrophilic reductive agents such as aluminium hydride or borane/THF complex (see Yardley et al., J. Med. Chem., method B).

Before precipitation, the solution of venlafaxine base can be advantageously treated with a suitable adsorber, such as activated carbon, silica gel, to remove a substantial part of colored impurities.

After the precipitation, isolation and/or drying of the solid venlafaxine base, the solid can be further processed by various known pharmaceutical schemes. For example, the solid can be milled if desired or used to form another compound or salt of venlafaxine. In this regard, the precipitation step can be used as a purification step in any venlafaxine-associated synthesis.

Venlafaxine base prepared in solid state may be, if necessary, further purified to the desired degree of purity or its crystalline properties may be improved by means of crystallization from a solution. The induced precipitation conditions as described previously may be used to (re)crystallize the venlafaxine base solid material. In particular, a preferred solvent system comprises water/ethanol mixture or ethyl acetate/heptane mixture. Crystallization is usually performed by lowering the temperature of the solution. Further, crystallization may be advantageously induced by a seeding crystal of venlafaxine base, especially in these preferred solvent systems.

In all of the precipitation and/or crystallization steps mentioned above, the temperature of the venlafaxine solution can be controlled to provide advantageous or desired results. Preferably the temperature is decreased during precipitation or crystallization. A faster decrease in temperature tends to form smaller particles of solid venlafaxine while a slower decrease tends to provide for larger particle sizes and/or higher bulk densities. To obtain crystalline venlafaxine base in a population of small particles it is preferred that the cooling rate be at least 20° C./hour. Alternatively, to obtain higher bulk density and/or larger particles it is preferred that the cooling rate be not greater than 20° C./hour. These cooling rates are preferred generally whether or not a seed crystal, a contrasolvent, or both are present. To produce large crystals such as those having an average size exceeding 250 microns the preferred cooling rate is about 10° C./hour. The cooling rate can be constant or variable. When variable cooling conditions are used, the average cooling rate should meet the recited range, e.g. not greater than 20° C./hour, more preferably, all of the cooling conditions used should meet fall within the not greater than 20° C./hour rate.

The venlafaxine base may be isolated in an anhydrous form or in a solvated, particularly hydrated form. Examples are venlafaxine hemihydrate, monohydrate, dihydrate, trihydrate, ethanol solvate, ethyl acetate solvate, acetone solvate or hexane solvate. The hydrates or solvates may be converted to preferred solvent-free or anhydrous form of venlafaxine base by conventional methods, e.g. by drying at reduced pressure. The anhydrate of the venlafaxine base precipitate forms a particular aspect of the invention.

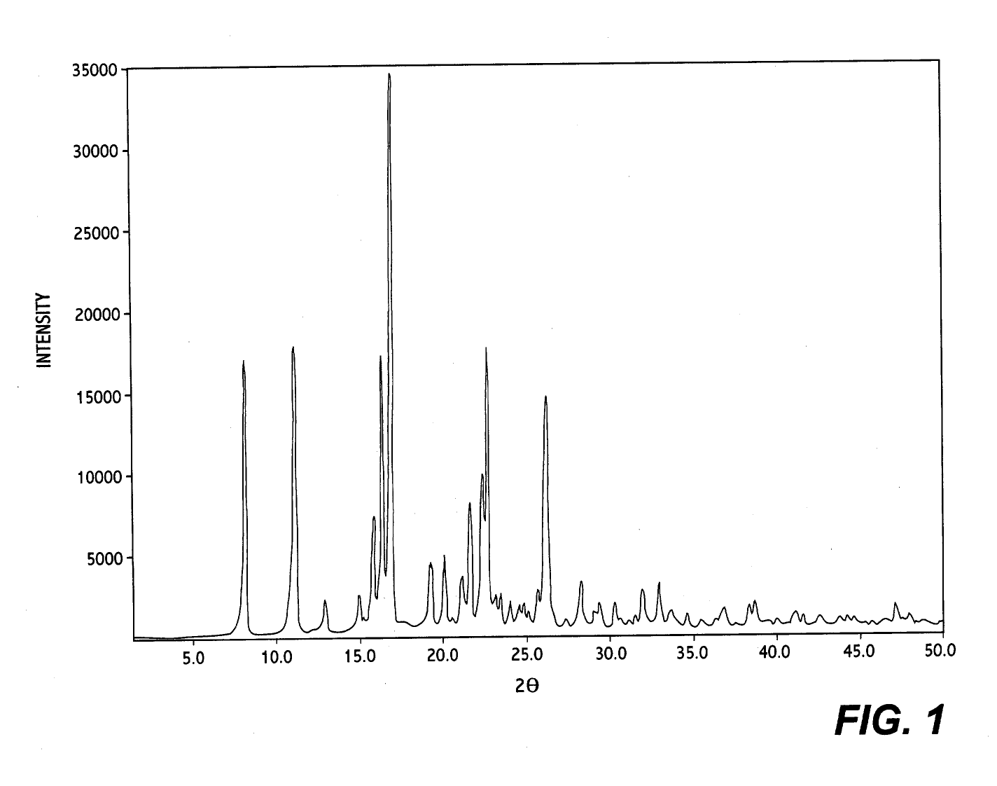

In particular, crystalline venlafaxine base may be isolated in various solid-state modifications. Frequently, the crystalline venlafaxine base is Form I. A substantially pure crystalline form has a powder X-ray diffraction pattern as shown in FIG. 1. Under differential scanning calorimetry (DSC), this substance exhibits a melting endotherm at approximately 75° C., an exotherm at approximately 78° C. and a second melting endotherm peak at approximately 80° C., measured at 1° C./min.

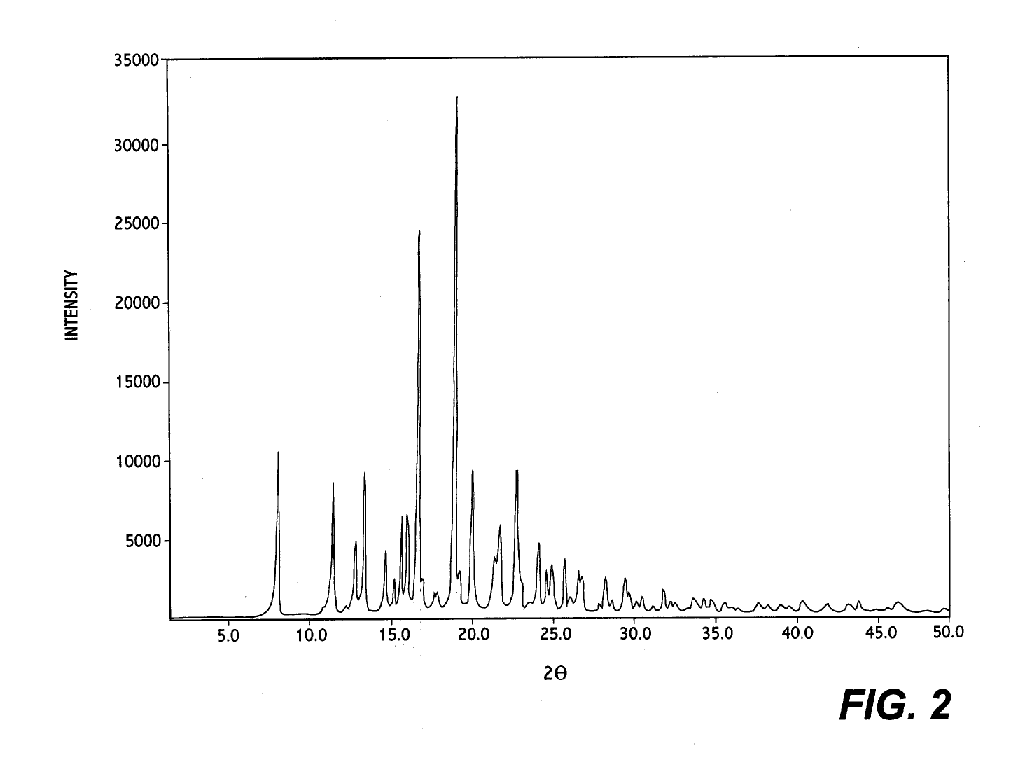

Furthermore, a Form II, differing from Form I, may be isolated as well. A substantially pure crystalline Form II has a powder X-ray diffraction pattern as shown in FIG. 2. Under differential scanning calorimetry (DSC), this substance exhibits a first melting endotherm at approximately 82° C., measured at 1° C./min.

In general Form II can be identified or distinguished from Form I via DSC, X-ray, or IR analysis. For example, a solid venlafaxine base that exhibits two melting endotherms under DSC analysis is, or contains, Form I. Because the second melting endotherm substantially corresponds to the melting endotherm of Form II, such a result does not exclude the presence of Form II. However, a solid venlafaxine base that exhibits a single melting endotherm generally substantially corresponds to Form II. It should be noted that while relatively pure Form II has a single melting endotherm at about 82° C., less pure samples may have a slightly lower peak such as at 80° C. Nonetheless, a single melting endotherm, even if shifted somewhat lower, is generally indicative of Form II venlafaxine. On a more coarse scale, Form II has a slightly higher melting point than Form I, but such a distinction can be obfuscated by impurities. According, the DSC measurement and the detection of one or two melting endotherms is a more accurate indicator of Form II versus Form I, respectively.

Regarding X-ray analysis, a solid venlafaxine base that exhibits peaks at angles of 2θ of 12.8, 13.3, 18.9, and 20.0 degrees +/−0.2 indicates that the solid is, or contains, venlafaxine Form II. These characteristic peaks are typically substantial peaks, meaning that they have an intensity of at least 10% of the maximum intensity, A solid venlafaxine that exhibits such peaks (at such intensity) under X-ray powder diffraction at least contains venlafaxine Form II. The solid may also contain venlafaxine Form I. Generally a mixture of forms will cause these peaks to be less intense than they would otherwise be in a substantially pure sample as shown in FIG. 2. Indeed, a significant amount of Form I can cause these peaks to be less than 10%, such as at least 5% of the maximum. Other characteristic peaks for identifying either Form I or Form II can be determined qualitatively by review of FIGS. 1 and 2 and/or more analytically from review of the following peak lists comprising some of the principal peaks.

Selected XRPD Peak List of Venlafaxine Base Form I

| angle 2θ | I/I max |
|---|---|
| (deg) | (%) |
| 8.200 | 100.00 |
| 11.170 | 76.68 |
| 12.950 | 7.54 |
| 15.010 | 7.02 |
| 15.850 | 21.71 |
| 16.370 | 50.45 |
| 16.940 | 98.28 |
| 19.200 | 10.24 |
| 19.365 | 9.56 |
| 20.105 | 11.30 |
| 21.140 | 7.72 |
| 21.630 | 17.82 |
| 22.345 | 21.06 |
| 22.680 | 36.46 |
| 26.025 | 14.36 |
| 26.275 | 26.76 |
| 28.300 | 5.28 |

Selected XRPD Peak List of Venlafaxine Base Form II

| angle 2θ | I/I max |
|---|---|
| (deg) | (%) |
| 8.000 | 72.68 |
| 11.410 | 40.67 |
| 12.785 | 20.57 |
| 13.330 | 38.15 |
| 14.620 | 16.28 |
| 15.135 | 8.73 |
| 15.605 | 23.76 |
| 15.965 | 24.14 |
| 16.655 | 84.41 |
| 16.940 | 7.47 |
| 18.890 | 100.00 |
| 19.220 | 8.34 |
| 19.975 | 26.21 |
| 21.330 | 9.89 |
| 21.700 | 15.22 |
| 22.685 | 23.49 |
| 23.035 | 5.08 |
| 24.050 | 11.11 |
| 24.525 | 6.64 |
| 24.895 | 7.65 |
| 25.675 | 8.02 |
| 26.515 | 6.10 |
| 26.755 | 5.31 |
| 28.190 | 4.98 |

Form II can also be identified by differences in IR spectra from Form I. The differences from Form I can be observed, inter alia, around 1240-1250 cm⁻¹ (peak reduced to small shoulder) 800-820 cm⁻¹ (additional side peak); 750 cm⁻¹ (peak reduced to small shoulder) in Form II.

The conditions of precipitating can control whether the resulting crystal is Form I or II. In general, Form II is preferentially formed if the precipitation occurs at enhanced temperatures, e.g. of about 50° C., while Form I is rather formed if the precipitation occurs at temperatures close to ambient or below. Further, using an ethanol/water mixture as the solvent generally yields Form I. Form II is preferentially formed by crystallization of venlafaxine base in the presence of Form II seeding crystals. Such seeding material can be obtained by precipitating venlafaxine base from a melt; i.e. melting venlafaxine and upon cooling allowing precipitation of solid venlafaxine crystals.

The solid venlafaxine base of the present invention can be a mixture of forms. For instance, a mixture of crystalline and non-crystalline forms or a mixture of crystalline forms. A mixture of Form I and Form II crystalline venlafaxine base is a specifically contemplated embodiment of the present invention. The relative amount of Form I and Form II is not particularly limited and includes from trace amounts to equal amounts to substantially pure amounts of either form. Generally the amount is within the range of 0.01:99.99 to 99.99:0.01, typically 1:99 to 99:1, more typically 5:95 to 95:5, for Form I to Form II, respectively. The mixture of forms can be deliberate such as by physical admixture or controlling the precipitation conditions, e.g. precipitating over multiple temperatures, etc., or accidental such as by conversion of one form to the other. Typically, the amount of one form is small relative to the other, i.e. the minor form is contained in an amount of 0.1% to 20%, more typically 0.2 to 10%, and even 1 to 10%. The minor form can be either Form I or Form II.

The solid, pharmaceutically useful venlafaxine base of the present invention is not limited to precipitates made by the above described induced precipitation method. Rather, the solid venlafaxine base of the present invention can be made by whatever means or source, provided that the solid is of sufficient quality to meet or exceed pharmaceutical standards. For example, solids made by evaporating off a solvent to form a colored residue, followed by, inter alia, washing, drying, and/or milling steps, are equally a part of the present invention provided that the solid possesses the necessary quality, i.e. it is pharmaceutically useful solid of venlafaxine base.

Solid venlafaxine base can advantageously be incorporated into a pharmaceutical composition by combining it with at least one pharmaceutically acceptable excipient. The pharmaceutical compositions of the present invention include the unit dosage form as well as the intermediate bulk formulations such as pellets, beads, powder blends, etc. Typically the composition is a finished dosage form also referred to as a unit dose. Dosage forms include oral dosage forms, topical dosage forms such as a transdermal patch, parenteral dosage forms such as an injectable solution, and rectal dosage forms such as a suppository, but is not limited thereto. Oral dosage forms are the most preferred due to the ease of administration and include solid oral dosage forms such as capsules, tablets, sachets/granules, and powders, as well as liquid oral dosage forms such as solutions, suspensions, and emulsions. Most preferred are solid oral dosage forms.

Pharmaceutically acceptable excipients are well known in the art and include diluents, fillers, binders, lubricants, disintegrants, glidants, colorants, pigments, taste masking agents, sweeteners, plasticizers, and any acceptable auxiliary substances such as absorption enhancers, penetration enhancers, surfactants, co-surfactants, and specialized oils. The proper excipient(s) are selected based in part on the dosage form, the intended mode of administration, the intended release rate, and manufacturing reliability. Examples of common types of excipients include various polymers, waxes, calcium phosphates, and sugars. Polymers include cellulose and cellulose derivatives such as HPMC, hydroxypropyl cellulose, hydroxyethyl cellulose, microcrystalline cellulose, carboxymethylcellulose, sodium carboxymethylcellulose, calcium carboxymethylcellulose, and ethylcellulose; polyvinylpyrrolidones; polyethylenoxides; and polyacrylic acids including their copolymers and crosslinked polymers thereof, i.e. Carbopol® (B. F. Goodrich), Eudragit® (Rohm), polycarbophil and chitosan polymers. Waxes include white beeswax, microcrystalline wax, carnauba wax, hydrogenated castor oil, glyceryl behenate, glycerylpalmito stearate, saturated polyglycolyzed glycerate. Calcium phosphates include dibasic calcium phosphate, anhydrous dibasic calcium phosphate, and tribasic calcium phosphate. Sugars include simple sugars such as lactose, maltose, mannitol, fructose, sorbitol, sacarose, xylitol, isomaltose, and glucose as well as complex sugars (polysaccharides) such as maltodextrin, amylodextrin, starches, and modified starches.

The venlafaxine base used in the pharmaceutical compositions of the present invention means any form of venlafaxine, derived from whatever source. However, the quality of the free base form must not be inconsistent with forming a pharmaceutical composition. When solid venlafaxine base is used in the composition, it is preferably one of the above-described pharmaceutically useful forms such as a white powder, precipitate, Form I, Form II, etc., but is not limited thereto.

The amount of venlafaxine base contained in a unit dosage form is an amount effective to treat one or more venlafaxine-treatable diseases or conditions as is hereinafter defined and can be determined by workers skilled in the art without undue experimentation. Generally this amount ranges from 2 mg to 300 mg. For oral dosage forms the amount is generally from 30 mg to 300 mg per unit dose. Contemplated doses include amounts of about 37.5 mg, 75 mg, 100 mg, 112.5 mg, 150 mg, 200 mg, and 300 mg strengths. Because the free base is used and not a salt thereof, the actual weight of the active ingredient is less in the present invention as compared with venlafaxine HCl, for example. This allows for higher loading of the active, i.e. more drug per weight of excipients, and therefore a lower amount of excipients are needed. Accordingly, a smaller dosage form is one advantage of using venlafaxine base. Preferably the dosage form contains at least 40 wt %, more preferably at least 50 wt % of the venlafaxine base.

It has been discovered that, due to the physical properties of venlafaxine base in terms of water solubility, an extended release tablet can be formed by simple techniques. In general, an otherwise immediate release tablet formulation, minus any disintegrant, provides for a multi-hour release of venlafaxine in water. Accordingly, an enteric coated tablet will provide for extended release of the venlafaxine. In a preferred embodiment, a multi-layer tablet is provided having an enteric coating around the tablet core and a further outerlayer above the enteric coating containing a minor amount of venlafaxine or a salt thereof. The outerlayer allows for immediate release of venlafaxine while the composition is in the acid environment of the stomach, while the majority of the venlafaxine is shielded from the acid by the enteric coating. This enteric coating allows for release of the venlafaxine in the core once the composition reaches the intestines and the corresponding higher pH. Generally no special extended release coating or matrix is needed to maintain the extended release as the insolubility of the venlafaxine base provides for slow release. Of course such release modifying agents can be incorporated if desired. Surprisingly, venlafaxine base can even be formulated into a once-a-day extended release tablet by such a simple design.

The tablets of venlafaxine base according to the present invention may be produced by any standard tabletting technique, e.g. by wet granulation, dry granulation or direct compression. The tabletting methods that do not employ a solvent (“dry processes”) are generally preferable.

Also, the venlafaxine base of the present invention can be formulated into traditional pellets, beads, and/or spheres. The pellets can be coated with an extended release coating or composition. In addition, different populations of coated pellets can be used in a single capsule, each providing a different release characteristic so that the aggregate release is sustained over a long period; i.e. 12 to 24 hours. Alternatively, the bead population can be substantially homogeneous. A preferred capsule of the pellet type is described in the above-mentioned U.S. Pat. No. 6,274,171 and related EP 0 797 991A1 wherein the venlafaxine hydrochloride used in these patents is replaced with the venlafaxine base of the present invention.

The venlafaxine base of the present invention can be used to treat any disease or condition that is treatable by venlafaxine. A venlafaxine-treatable disease or condition is one that could be improved by a serotonin or norepinephrine uptake inhibitor and specifically includes, without limitation, depressions, panic disorder, generalized anxiety disorder, obesity, post-traumatic stress disorder, late luteal phase dysphoric disorder, attention deficit disorders, Gilles de la Tourette syndrome, bulimia nervosa, and Shy Drager syndrome. See published U.S. patent application U.S. 2001/0012855 A1 for a description of the uses of venlafaxine and salts thereof. The venlafaxine base of the present invention can be used to treat such conditions by administering an effective amount to a patient in need thereof. An effective amount is generally known in the art and/or determined using routine skill. Typically the effective amount for a human is 30 to 300 mg of venlafaxine per day. The patients used herein include human and non-human mammals such as dogs, cats, and horses. The route of administration is not particularly limited and includes peroral, parenteral, and transdermal administration. Preferably, the venlafaxine base is administered orally via one or two unit dosage forms, especially extended release tablets or capsules, as described above.

The entire disclosure in each of the patents mentioned in the above description is incorporated herein by reference. The invention will be further described with reference to the following non-limiting examples. In the examples, racemic venlafaxine is used unless other indicated.

### REFERENCE EXAMPLE 1

In a 10 l flask, equipped with a mechanical stirrer,

| | | |
|---|---|---|
| 750 | g | of venlafaxine hydrochloride was suspended in a mixture of |
| 3 | l | water and |
| 3 | l | ethyl acetate. Then, |
| 357 | ml | ammonium hydroxide in water (28-30% aqueous) was added |
| | | while stirring. The stirring was continued for 30 minutes |
| | | and the layers were allowed to separate. The aqueous layer |
| | | was extracted with |
| 750 | ml | ethyl acetate. The combined organic layers were washed with |
| 1.5 | l | saturated aqueous NaCl, dried (Na2SO4), filtered and |
| | | concentrated at reduced pressure. The obtained white solid |
| | | was dried overnight at 40° C. in vacuo. |

The whole sequence was performed with another 750 g of venlafaxine HCl and the obtained material was combined with that of the preceded batch.

Total isolated yield was 1309.1 g (98.7%). ¹H-NMR confirmed the structure of the product.

### REFERENCE EXAMPLE 2

In a 100 ml flask, equipped with a stirring bar,

| | | |
|---|---|---|
| 2.5 | g | of 1-[2-amino-1-(4-methoxyphenyl)ethyl]cyclohexanol |
| | | (see J. Med. Chem. 33,2902(1990)) was suspended in |
| 23 | ml | water, |
| 2.4 | ml | 37% formaldehyde and |
| 3.2 | ml | formic acid were added successively. The reaction mixture |
| | | was refluxed for 18 hours and was subsequently cooled |
| | | (ice-water bath). Then, the pH was set to 10-11 by addition |
| | | of about |
| 10 | ml | 5 N aqueous NaOH. The obtained suspension was extracted |
| | | with |
| 3*20 | ml | ethyl acetate. The combined organic layers were washed with |
| | | brine and dried over Na₂SO₄. The resulting dried organic |
| | | layer was divided in two equal portions. |

Portion 1 was concentrated at reduced pressure to dryness to give a brown gum, which solidified upon standing at room temperature.

Yield: 1.164 g of a brown solid

Purity: 92%

¹H-NMR in agreement

Mp 65.0-71.5° C.

Determination of coloration of liquids according to Ph.Eur.2.2.2, method II was as follows:

0.100 gram of the sample was dissolved in 100 ml 1% aqueous HCl (m/V) and the solution was filtered (glass filter) before evaluated in the coloration test. The sample solution was compared with a coloration scale B1-B9 of Ph.Eur. 2.2.2. The sample showed a color of B7-B8.

### EXAMPLE 1A

In a 100 ml flask, equipped with a stirring bar,

| | | |
|---|---|---|
| 3.0 | g | racemic venlafaxine HCl was dissolved in |
| 8 | ml | water. Then, |
| 0.382 | g | sodium hydroxide (1 equivalent) was added in one portion. |
| | | Immediately, an oil separated from the reaction mixture. |
| 8 | ml | ethanol was added and the resulting mixture was seeded with |
| | | a few crystals After a few minutes, the oil had solidified, |
| | | but stirring was continued for 1 hour. The crystals were |
| | | isolated by filtration, washed with 100 ml of a mixture |
| | | of ethanol/water (¼), dried overnight at 40° C. under |
| | | vacuum. |

Yield: 2.276 g (86.9%).

mp (76-78° C.), Karl-Fischer (no water).

The solid state properties correspond to venlafaxine base Form I

### EXAMPLE 1B

In a 100 ml flask, equipped with a stirring bar,

| | | |
|---|---|---|
| 3.0 | g | racemic venlafaxine HCl was dissolved in |
| 8 | ml | ethanol. Then, |
| 0.382 | g | sodium hydroxide in |
| 6 | ml | water was added dropwise at room temperature while |
| | | stirring. Another |
| 2 | ml | water was used to transfer all the aqueous sodium hydroxide |
| | | into the reaction mixture. During addition, the reaction |
| | | mixture first became clear and then an oil separated from |
| | | the mixture. After 15 minutes, a few crystals were added |
| | | and the oil slowly solidified. Stirring was continued for |
| | | 30 minutes. The crystals were isolated by filtration, |
| | | washed with 10 ml of a mixture of ethanol/water (¼), |
| | | dried overnight at 40° C. under vacuum over P₂O₅. |

Yield: 2.207 g (84.3%).

mp (76.7-78.7° C.), Karl-Fischer (no water).

### EXAMPLE 1C

In a 100 ml flask, equipped with a stirring bar,

| | | |
|---|---|---|
| 5.0 | g | venlafaxine HCl was dissolved in |
| 20 | ml | water and |
| 20 | ml | ethyl acetate. Then, |
| 2.4 | ml | 28-30% aqueous ammonium hydroxide was added while |
| | | stirring. The stirring was continued for 30 minutes and the |
| | | layers were allowed to separate. The aqueous phase was |
| | | extracted with |
| 5 | ml | ethyl acetate. The combined organic layers were washed with |
| 10 | ml | brine, dried (Na₂SO₄), filtered, and concentrated at reduced |
| | | pressure to a volume of ˜6 ml. Then, |
| 20 | ml | heptane was added and the resulting suspension was stirred. |
| | | After a few minutes, crystals appeared and the mixture was |
| | | stirred for another 30 minutes at room temperature and 30 |
| | | minutes at 4° C. The crystals were isolated by filtration , |
| | | washed with heptane and dried overnight at 40° C. under |
| | | vacuum. |

Yield: 2.397 g (55%).

mp (76.4-78.4° C.), Karl-Fischer (no water).

### EXAMPLE 1D

In a 100 ml flask, equipped with a stirring bar,

| | | |
|---|---|---|
| 5.0 | g | venlafaxine besylate monohydrate was suspended at room |
| | | temperature in |
| 10 | ml | ethanol. Then, |
| 0.441 | g | sodium hydroxide in |
| 10 | ml | water was added slowly. To the resulting clear solution, a few |
| | | crystals (<5 mg) venlafaxine base were added in order to |
| | | initiate the crystallization process. The reaction mixture was |
| | | stirred at room temperature for another 2 hours. The crystals |
| | | were isolated by filtration, washed with |
| 10 | ml | water, and dried overnight at 40° C. under reduced pressure. |

Isolated yield of venlafaxine base: 2.539 g (83%).

Mp.=77.2-79.2° C., Karl-Fischer: 0.06% water (uncorrected)

DSC: peak at 79.93° C.

### EXAMPLE 1E

In a 1 l flask,

| | | |
|---|---|---|
| 100 | g | of venlafaxine hydrochloride was suspended in |
| 275 | ml | ethanol. Then, a solution of |
| 12.73 | g | sodium hydroxide in |
| 200 | ml | water was added in 5 minutes at room temperature while |
| | | stirring mechanically. When half of the amount of aqueous |
| | | NaOH was added, a few seeding crystals of venlafaxine |
| | | base were added. However, the crystallization did not start |
| | | until all aqueous NaOH was added. After complete |
| | | addition, the temperature of the mixture had risen to 30° C. |
| | | Another |
| 75 | ml | water was used for transporting the aqueous NaOH into the |
| | | reaction flask. After stirring for 2-2.5 hours at room |
| | | temperature, the crystals were isolated by filtration and |
| | | washed with |
| 3*100 | ml | water. The crystals were dried overnight at 40° C. under |
| | | vacuum. |

Isolated yield was 68.9 g (79%) of high purity venlafaxine Form I.

### EXAMPLE 2A

In a 100 ml flask, equipped with a stirring bar,

| | | |
|---|---|---|
| 5.0 | g | crude venlafaxine base (prepared as in Reference |
| | | example 1, i.e. neutralization of venlafaxine HCl and |
| | | extraction with ethyl acetate and subsequently concentration |
| | | to dryness) was dissolved at room temperature in |
| 15 | ml | ethanol. Then, to the clear solution, |
| 15 | ml | water was added to form a white oily suspension. After |
| | | stirring for 15 minutes, a few crystals (<5 mg) venlafaxine |
| | | base were added to initiate the crystallization process. The |
| | | reaction mixture was stirred at room temperature for another 2 |
| | | hours. The crystals were isolated by filtration, washed with |
| 10 | ml | water, and dried overnight at 40° C. in vacuo. |

Isolated yield of venlafaxine base: 4.255 g (85.1%).

IR: corresponds to Form I

Mp.=76.8-77.8° C., Karl-Fischer: 0.06% water (uncorrected)

DSC: peak at 79.30° C.

### EXAMPLE 2B

Portion 2 of the solution obtained in Reference example 2 was concentrated to a volume of ˜2 ml. Then,

| | | |
|---|---|---|
| 6 | ml | n-heptane was added and the resulting suspension was heated |
| | | into solution. The hot, clear yellow/orange solution was allowed |
| | | to cool to room temperature, while stirring, and then further |
| | | cooled to 4° C. The crystals were isolated by filtration, washed |
| | | with |
| 2 | ml | n-heptane, and dried in vacuum overnight at 40° C. |

Yield: 0.309 g off-white solid

Purity: 95.5% (HPLC)

IR corresponds with Form I

Mp. 71.0-76.0° C.

Determination of coloration of liquids according to Ph.Eur.2.2.2, method II, corresponded to B₉.(=colorless)

### EXAMPLE 3

In a 100 ml flask, equipped with a stirring bar,

| | | |
|---|---|---|
| 3.0 | g | of 1-[cyano(4-methoxyphenyl)methyl]cyclohexanol was |
| | | suspended in |
| 25 | ml | 7 N NH₃ solution in methanol. Then, |
| 500 | mg | Raney-Cobalt (pre-washed in methanol) was added. The |
| | | mixture was hydrogenated at 50 bar hydrogen gas at 50° C. |
| | | for 6 hours. After cooling to room temperature, the suspension |
| | | was filtered over Celite and evaporated under reduced |
| | | pressure. A brown oil was obtained. Yield: 3.0 gram. |
| | | NMR confirmed the structure of 1-[2-amino-1-(4- |
| | | methoxyphenyl)ethyl]cyclohexanol. Then, |
| 28 | ml | water, |
| 2.9 | ml | 37% formaldehyde and |
| 3.9 | ml | formic acid were added successively. The reaction mixture |
| | | was refluxed for 1 night and was subsequently cooled (ice- |
| | | water bath) and the pH was set to 10-11 by addition of about |
| 10 | ml | 5N aqueous NaOH. The obtained suspension was seeded with |
| 20 | mg | venlafaxine base and the resulting suspension was stirred for |
| | | 1 hour. The solid was isolated by filtration, was washed with |
| 2*5 | ml | water, and was dried in a vacuum oven at 40° C. overnight. |

Yield: 2.45 gram venlafaxine base Form I

Purity: 95.56% (HPLC) EXAMPLE 4A

In a 100 ml flask, equipped with a stirring bar,

| | | |
|---|---|---|
| 10.0 | g | crude venlafaxine base was suspended at room temperature in |
| 15 | ml | n-heptane and |
| 4 | ml | ethyl acetate. The suspension was heated into solution and the |
| | | resulting hot, clear solution was allowed to cool to room |
| | | temperature overnight. The crystals (white crystalline) were |
| | | isolated by filtration, washed with |
| 2 | ml | n-heptane, and dried at 40° C. in vacuo for 1 day. |

Isolated yield of venlafaxine base: 7.371 g (73.7%).

¹H-NMR (3c7633b1): confirmed the expected structure

IR: corresponds to Form I

Mp.=76.8-77.5° C., Karl-Fischer: 0.06% water (uncorrected)

DSC: peak at 80.28° C.

### EXAMPLE 4B

In a 100 ml flask, equipped with a stirring bar,

| | | |
|---|---|---|
| 2.4 | g | crude venlafaxine base was dissolved/suspended at room |
| | | temperature in |
| 50 | ml | ethyl acetate. Some insoluble particles were removed by |
| | | filtration over Celite. The filtrate was concentrated to a |
| | | volume of ˜2 ml, followed by addition of |
| 5 | ml | n-heptane. After storage for 4 hours at 4° C., nice crystals had |
| | | formed, which were isolated by filtration. |

Isolated yield of venlafaxine base (white crystalline material): 1.631 g (68.0%).

Purity (HPLC): 98.16%

IR: corresponds to Form I

Mp.=74.2-79.8° C.

### EXAMPLE 5

In a 1 l flask,

| | | |
|---|---|---|
| 130 | g | venlafaxine base was heated into solution using |
| 250 | ml | ethanol and |
| 200 | ml | water. Upon cooling to room temperature, an oil was formed, |
| | | which after a few hours started to solidify. After storage at |
| | | room temperature overnight, a large amount of white crystals |
| | | had formed, which were isolated by filtration and washed with |
| | | 100 ml of a mixture of ethanol/water (¼). The crystals were |
| | | dried overnight at 40° C. under vacuum. |

Isolated yield was 127 g (98%). DSC (peak at 79.77° C.), mp (=77-79° C.), purity on HPLC (>99.8%). Karl-Fischer: no water present.

More than 95% of particles have particle size from 0.5 to 200 microns.

### EXAMPLE 6

### Venlafaxine Form II from Melt

In a 100 ml flask (closed),

| | | |
|---|---|---|
| 2 | g | venlafaxine base was heated using an oil bath to a temperature of |
| | | 90-120° C. venlafaxine melted rapidly (within a few minutes). |
| | | The flask was kept in the oil bath for another 5 minutes. Then, |
| | | the flask was stored at room temperature. Initially, a colorless |
| | | glassy substance was formed, which upon standing at room |
| | | temperature changed into a white crystalline material. |

IR: corresponds with Form II

### EXAMPLE 7A

### Form II by Crystallization

In a 100 ml flask,

| | | |
|---|---|---|
| 0.50 | g | venlafaxine base dissolved in |
| 1 | ml | ethanol was added dropwise under stirring to |
| 20 | ml | water at a temperature of 75° C. The ethanol solution was |
| | | filtered before the addition. The clear solution was seeded |
| | | with Form II (Ex 6). The heating was turned off and, still in |
| | | the oil bath, the solution was allowed to cool to room |
| | | temperature slowly. The thus obtained solid was isolated by |
| | | filtration and dried over P₂O₅ at room temperature in vacuum. |

Isolated yield: 0.258 g (51.6%).

IR: corresponds to Form II.

DSC: peak at 82.22° C. Example 7B

In a 500 ml flask,

| | | |
|---|---|---|
| 10 | g | venlafaxine base was dissolved in |
| 25 | ml | ethanol, filtered prior to addition, and added dropwise to |
| 200 | ml | water at 70° C. while stirring mechanically. During and after |
| | | addition, seed crystal of venlafaxine Form II was added in |
| | | trace amounts. After complete addition of the ethanol solution, |
| | | the temperature of the oil bath was lowered to 50° C. At this |
| | | temperature, crystallization occurred. After an additional hour |
| | | at 50° C., the crystals were isolated by filtration and |
| | | dried over P₂O₅ at 25° C. overnight. |

Isolated yield: 9.312 g (93%).

IR: corresponds to Form II

DSC: single peak at 82.61° C.

### EXAMPLE 8A

In a 500 ml flask,

| | | |
|---|---|---|
| 50 | g | venlafaxine free base was dissolved in |
| 270 | ml | ethyl acetate and washed with |
| 90 | ml | water. The organic phase was dried (Na₂SO₄) and filtered, |
| | | (the solid residue was washed with |
| 20 | ml | ethyl acetate), and concentrated using a Dean-Stark apparatus |
| | | (oil bath 120° C.) to approx. 50 ml. Then, |
| 300 | ml | heptane was added and another 100 ml of liquids was |
| | | removed by distillation (Dean-Stark). The remaining hot |
| | | solution (250 ml) was allowed to cool at a cooling rate |
| | | 10° C./hour while stirring mechanically (200 rpm). At 35° C., |
| | | seeding crystals of venlafaxine base Form I were added to |
| | | initiate the crystallisation process and the cooling continued. |
| | | After stirring at 14° C. for 1 hour, the crystals were isolated |
| | | by filtration, washed with |
| 20 | ml | cold heptane, and dried overnight at 40° C. in vacuo. |

Yield: 35.3 g (71%) of venlafaxine base

Particle size: <500 μm

### EXAMPLE 8B

In a 500 ml flask,

| | | |
|---|---|---|
| 50 | g | venlafaxine base was dissolved in |
| 270 | ml | ethyl acetate and washed with |
| 90 | ml | water. The organic phase was dried (Na₂SO₄), filtered (the |
| | | solid residue was washed with |
| 20 | ml | ethyl acetate), and concentrated using a Dean-Stark apparatus |
| | | (oil bath 110-120° C.). to approx. 50 ml. Then, |
| 100 | ml | heptane was added and another 100 ml was removed by |
| | | distillation (Dean-Stark). To the remaining 50 ml solution, |
| 200 | ml | heptane was added and the resulting solution was allowed to |
| | | cool while stirring mechanically (300 rpm). At approx. |
| | | 35° C., seeding crystals of venlafaxine base Form I were |
| | | added and the crystallisation process started. After stirring |
| | | for another 1.5 hours, the crystals were isolated by filtration, |
| | | washed with |
| 20 | ml | heptane, dried overnight at 40° C. in vacuo. |

Yield: 30.02 g (60%) of venlafaxine base

Particle size: <200 μm

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

We claim:

1. A crystalline racemic venlafaxine base wherein said venlafaxine is in particle form having an average particle size within the range of 0.5 to 200 microns.

2. The venlafaxine according to claim 1, wherein said crystalline venlafaxine base has an average particle size within the range of 10 to 100 microns.

3. The venlafaxine according to claim 1, wherein said venlafaxine contains 2.0 wt % or less of impurities.

4. The venlafaxine according to claim 1, wherein said crystalline venlafaxine was formed by precipitation from a solution containing venlafaxine or a salt thereof.

5. The venlafaxine according to claim 1, which is substantially free of ethyl acetate.

6. A crystalline venlafaxine base in particle form having a bulk density of at least 0.4 mg/ml.

7. The crystalline venlafaxine according to claim 6, wherein said venlafaxine has a bulk density within the range of 0.4 to 0.8 mg/ml.

8. The venlafaxine according to claim 6, wherein said venlafaxine contains 2.0 wt % or less of impurities.

9. The venlafaxine according to claim 6, wherein said crystalline venlafaxine was formed by precipitation from a solution containing venlafaxine or a salt thereof.

10. The venlafaxine according to claim 6, which is substantially free of ethyl acetate.

11. A venlafaxine base in solid form and exhibiting an x-ray diffraction pattern that includes peaks at angles of 2θ of 12.8, 13.3, 18.9, and 20.0 degrees +/−0.2, said peaks having an intensity of at least 10% of the maximum intensity.

12. The venlafaxine according to claim 11, wherein said venlafaxine is in the form of a white powder.

13. The venlafaxine according to claim 11, wherein said venlafaxine is at least 98% pure.

14. A venlafaxine solid comprising a mixture of crystalline venlafaxine base of forms I and II.

15. The venlafaxine solid according to claim 14, wherein the amount of venlafaxine base of Form I comprises 80% to 99.9% of the solid.

16. The venlafaxine solid according to claim 14, wherein the amount of venlafaxine base of Form II comprises 80% to 99.9% of the solid.

17. A process for making solid racemic venlafaxine base, which comprises precipitating racemic venlafaxine base from a solution of venlafaxine to form crystalline racemic venlafaxine base; wherein said precipitation is carried out in accordance with at least one of the following conditions:
(i) said solution contains a combination of solvents comprising at least one organic polar solvent and at least one contrasolvent selected from water, an aliphatic hydrocarbon and an alicyclic hydrocarbon; or
(ii) a seeding crystal of venlafaxine base is present.

18. The process according to claim 17, wherein said organic polar solvent is selected from the group consisting of ethyl acetate, diethyl ether, acetone, methanol, ethanol, and propanol.

19. The process according to claim 18, wherein said contrasolvent is selected from the group consisting of hexane, heptane, petroleum ether, and cyclohexane.

20. The process according to claim 17, wherein said precipitating is carried out under condition (i) and said solvent is a mixture of ethanol or ethyl acetate with n-heptane.

21. The process according to claim 17, wherein said precipitating is carried out under condition (i) and said solvent is a mixture of ethanol and water.

22. The process according to claim 17, wherein said precipitating step is carried out under condition (ii).

23. The process according to claim 22, wherein said seed crystal is venlafaxine Form I.

24. The process according to claim 22, wherein said seed crystal is venlafaxine Form II.

25. The process according to claim 17, wherein said precipitated venlafaxine base has an average particle size within the range of 0.5 to 200 microns and at least 50% of the particles are within the range of +/−25 microns from the average particle size.

26. A process for making crystalline venlafaxine base of bulk density of at least 0.4 mg/ml, which comprises crystallizing venlafaxine base from a solvent while cooling, wherein the cooling rate is not greater than 20° C./hour.

27. The process according to claim 26, wherein said cooling rate is about 10° C./hour.

28. The process according to claim 26, wherein said crystallization is carried out in the presence of a seed crystal.
